(19) United States
(12) Patent Application Publication
(10) Pub. No.: US 2017/0100552 A1
(43) Pub. Date: Apr. 13, 2017

(54) CONTROL AND DISPLAY AREA FOR A MEDICAL VENTILATOR

(71) Applicant: Loewenstein Medical Technology S.A., Luxembourg (LU)

(72) Inventor: Anne WONSYLD, Hamburg (DE)

(21) Appl. No.: 15/287,939

(22) Filed: Oct. 7, 2016

(30) Foreign Application Priority Data
Oct. 8, 2015 (DE) 102015012996.1

(51) Int. Cl. A61M 16/00

(52) U.S. Cl. CPC A61M 16/0003; A61M 2205/505; A61M 2207/00

(57) ABSTRACT

Disclosed is a control and display area for a medical ventilator, comprising or consisting of a control film having at least one control element. The control film is optically transparent in one or more sections thereof and opaque in one or more other sections thereof and at least the optically transparent section(s) of the control film is/are connected by an optically clear adhesive element with a display lying therebelow and the one or more opaque sections, at least in part, is/are connected by an adhesive element with the housing of the medical ventilator lying therebelow.

### CROSS-REFERENCE TO RELATED APPLICATIONS

The present application claims priority under 35 U.S.C. §119 of German Patent Application No. 10 2015 012 996.1, filed Oct. 8, 2015, the entire disclosure of which is expressly incorporated by reference herein.

### BACKGROUND OF THE INVENTION

1. Field of the Invention

The present invention relates to a control and display area for a medical ventilator.

2. Discussion of Background Information

Medical ventilators have visual display systems to display the operating state of the device or further information. Moreover, medical ventilators have at least one control element.

Here, the display and the control elements are embedded in the housing of the medical ventilator. Hence, there are necessarily joints in the control and display area in the transition zone between the display and the housing and in the transition zone between the control element and the housing.

By way of example, hygienic preparation or cleaning of medical ventilators in hospitals provides for a daily wipe disinfection of the device surfaces, or else spray disinfection.

The joints in the housing must therefore be sealed in such a way that moisture cannot ingress into the medical ventilator. Moreover, there must not be accumulation of dirt at the transitions since this can be fertile ground for bacteria. The structural measures required for this increase the production and assembly costs.

In view of the foregoing, it would be advantageous to have available a control and display area for a medical ventilator which is easy to clean and can be produced and assembled in a cost-effective manner. It further would be advantageous to have available a method for producing a control and display area for a medical ventilator which is easy to clean and can be produced and assembled in a cost-effective manner.

### SUMMARY OF THE INVENTION

The present invention provides a control and display area for a medical ventilator, comprising or consisting of a control film with at least one control element, the control film being optically transparent at least in one or more sections and opaque at least in one or more other sections. At least the optically transparent section(s) of the control film is/are connected by an optically clear adhesive element with a display lying therebelow and the opaque section(s), at least in part, is/are connected by an adhesive element with the housing of the medical ventilator lying therebelow.

The control and display area may also be characterized in that the display is an LED display or an LCD display with a glass-pane surface.

The control and display area is preferably characterized in that a touch film with a transparent electrode layer is arranged between the film and the LED display unit.

The control and display area may also be characterized in that the adhesive element is or comprises an optically clear double-sided adhesive tape with a light transmission of greater than 95%.

The adhesive element preferably is an optically clear double-sided adhesive tape with a light transmission of greater than 98%.

The control and display area may also be characterized in that the optically clear double-sided adhesive tape is arranged over the whole area between display and film.

The control and display area may also be characterized in that the optically clear double-sided adhesive tape comprises an acrylate adhesive.

The control and display area may also be characterized in that the adhesive element is an optically clear adhesive which has a light transmission of greater than 95% and fills the space between the film and the display.

The control and display area may also be characterized in that the adhesive element is an optically clear adhesive which has a light transmission of greater than 98% and fills the space between the film and the display without air bubbles.

The control and display area may also be characterized in that the control film has a multi-layered design and comprises, at least in part, polyester and polycarbonate and conductor track structures for the control elements and has a thickness of at most about 3 mm.

The control and display area may also be characterized in that the control film is adhesively bonded, over the whole area thereof, onto the surface of the medical ventilator pointing to the user.

The control and display area may also be characterized in that the control film covers more than about 70% of the surface of the medical ventilator pointing to the user.

The control and display area may also be characterized in that the control film covers more than about 80% of the surface of the medical ventilator pointing to the user.

The control and display area may also be characterized in that the control film covers more than about 90% of the surface of the medical ventilator pointing to the user.

The control and display area may also be characterized in that the control film covers the entire surface of the medical ventilator pointing to the user.

The control and display area may also be characterized in that the area of the control film is at least about twice the size of the area of the display.

The method for producing a control and display area for a medical ventilator, comprising or consisting of a control film with at least one control element, is characterized in that the control film is optically transparent at least in one or more sections and opaque at least in one or more other sections, and that at least the optically transparent section(s) of the control film is/are connected by an optically clear adhesive element with a display lying therebelow and that the opaque section(s), at least in part, is/are connected by an adhesive element with the housing of the medical ventilator lying therebelow.

Usually, polymer panes (e.g. made of polycarbonate, polymethyl methacrylate, etc.) or glass panes are used as protective window for the display.

The double-sided adhesive tapes are matched to the material to be adhered; thus, for adhering glasses, use is usually made of adhesive tapes which exhibit a certain shock absorption behavior and are therefore able to buffer mechanical impulses, whereas the compensation of bending stresses is more likely to be important when adhering plastics (polymer panes).

“Optical bonding” means “transparent liquid adhesive bonding” and describes an adhesive technology, by means of which optical components, such as e.g. displays, touch or front glass and film, are connected to one another with particularly high optical quality. What is ensured in the production process by way of completely casting the interstices is that there are no air inclusions between display element and front glass such that condensation effects may be completely precluded. Filling the air gap moreover prevents parallax errors and internal reflections, which leads to an improved optical performance in the case of disadvantageous daylight conditions.

A further advantage lies in the brilliance which, in particular, is obtained in conjunction with LED displays.

In addition to all the optical advantages as a result of optical bonding, the robustness of the overall design is advantageous for the rough use in industry. The components securely connected to one another are very resistive to vibrations, thermal tensions and loads due to shock.

One of the largest challenges lies in adhering together optical materials without air bubbles. Where liquids come into contact with air, air inclusions may also arise as a matter of principle, said air inclusions then being distributed in the liquid in the form of air bubbles. Depending on the viscosity and surface tension of the adhesive, the air bubbles disappear at the surface. However, when using liquid adhesive for joining together film and glass plate, it is once again possible for air bubbles to be included and these then can no longer escape during the further course of the adhering process and remain included between the optical joining partners.

If, initially, the multiplicity of employed adhesives are ignored, it is possible, in principle, to distinguish between two different techniques for the bonding methods: the displacement method and the filling method. In the case of the displacement method, display and touch component are connected to one another with a liquid adhesive, excess material can be wiped away at the outer edge. A further known method is one where the components to be adhered are initially positioned in relation to one another and the air gap is subsequently filled with liquid adhesive.

According to the invention, a film is used as carrier material, said film being laminated, over the whole area thereof, onto the rear side of the front pane of the display.

As a result of using a touch film instead of a conventional touchscreen, a glass layer between display and cover glass is dispensed with.

### BRIEF DESCRIPTION OF THE DRAWINGS

Further advantages and features of the present invention result from the description of the exemplary embodiment, which is explained hereafter with reference to the appended drawings.

In the drawings:

FIG. 1 shows a control and display area for a medical ventilator; and

FIG. 2 shows a section through the control and display area for a medical ventilator.

### DETAILED DESCRIPTION OF EXEMPLARY EMBODIMENT

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

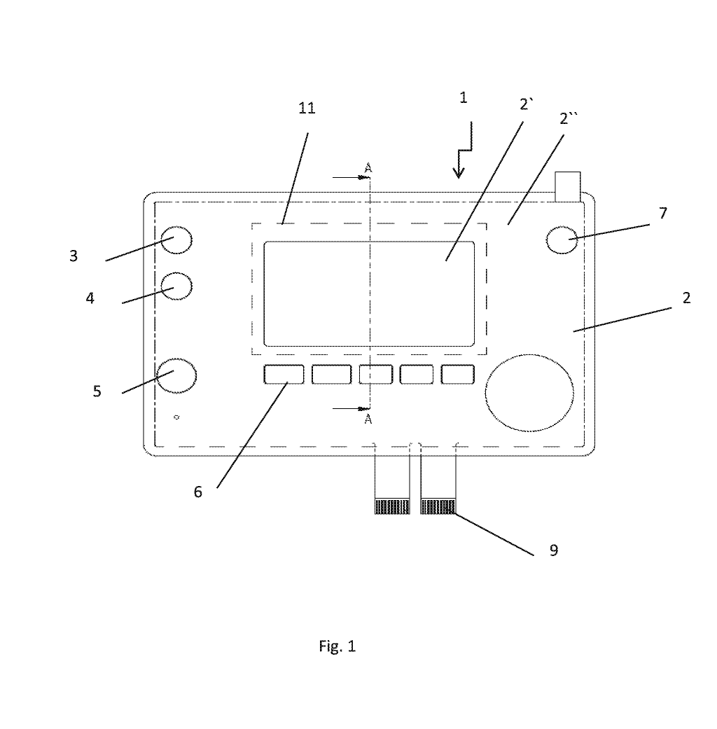

FIG. 1 shows a control and display area (1) for a medical ventilator, consisting of a control film (2) with at least one control element (3-7), wherein the control film (2) is optically transparent (2′) at least in sections and opaque (2″) at least in sections, wherein at least the optically transparent section (2′) of the control film (2) is connected by an optically clear adhesive element (10′) with a display (11, 12) lying therebelow and wherein the opaque section (2″), at least in part, is connected by an adhesive element with the housing of the medical ventilator (20) lying therebelow. By way of example, the display (11) is an LED display with a pane surface (12). A touch film (13) with a transparent electrode layer may be arranged between the film (2) and the LED display unit (11).

The control film (2) preferably has a multi-layered design and comprises, at least in part, polyester and polycarbonate and conductor track structures (9) for the control elements and a thickness of at most about 3 mm. By way of example, the control film (2) is adhesively bonded, over the whole area thereof, onto the surface of the medical ventilator (20) pointing to the user and covers more than about 70% of the surface of the medical ventilator pointing to the user, or e.g. covers the entire surface. The area of the control film (2) is at least twice the size of the area of the display (11).

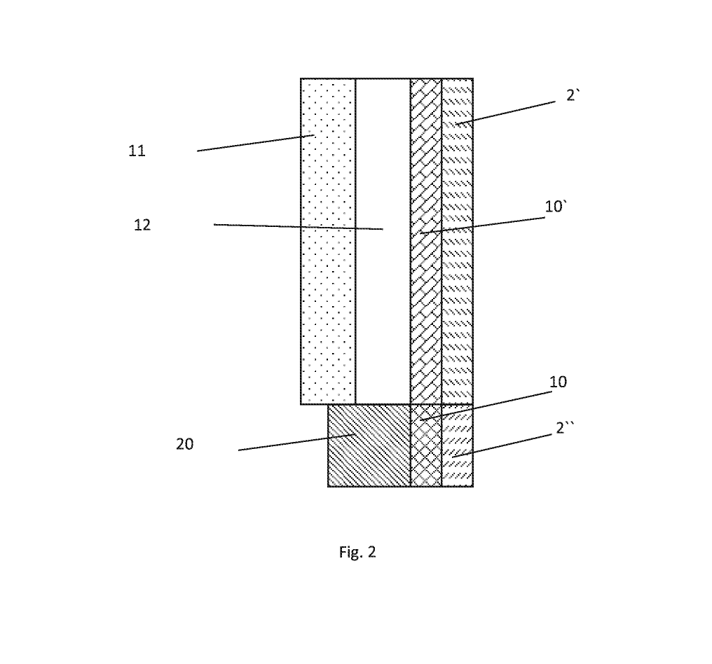

FIG. 2 shows a section through the control and display area (1) for a medical ventilator, consisting of a control film (2) with at least one control element (3-7), wherein the control film (2) is optically transparent (2′) at least in sections and opaque (2″) at least in sections, wherein at least the optically transparent section (2′) of the control film (2) is connected by an optically clear adhesive element (10′) with a display (11, 12) lying therebelow and wherein the opaque section (2″), at least in part, is connected by an adhesive element (10), which may be optically clear or, for example, be a liquid adhesive or a double-sided adhesive tape, with the housing of the medical ventilator (20) lying therebelow. By way of example, the display (11) is an LED display with a pane surface (12). A touch film (13) with a transparent electrode layer may be arranged between the film (2) and the LED display unit (11).

The control film (2) preferably has a multi-layered design and comprises, at least in part(s), polyester and polycarbonate and conductor track structures (9) for the control elements and a thickness of at most about 3 mm.

As a result of using the control film (2) and the extensive adhering, the control and display area (1) serves, firstly, to protect against the ingress of moisture and, secondly, to protect against damage to the display due to vibrations, thermal tensions and loads due to shock.

As a result of using the control film (2) for the control and display area (1), which is shown as an integral embodiment, but may have a multi-layered design, provision is made of a continuous surface without abrupt transitions or edges. Thus, the integral control film provides a continuous, i.e. uninterrupted in space, surface of the control and display area. Moreover, the control film is resistive to disinfection means. Therefore, it is easy to be able to clean it. No joints or slits or edges, at which dirt or bacteria can accumulate, arise. In the region of the control elements (3-7), the control and display area (1), as a result of using conductor track structures (9), provides a surface which has only slightly elevated areas with a very slight elevation and large run-off radii in the region of the control elements (3-7).

The control elements (3-7) are identifiable not only optically but also in a tactile manner as a result of the slight elevations.

While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A control and display area for a medical ventilator, wherein the area comprises or consists of a control film having at least one control element, the control film being optically transparent in one or more sections thereof and opaque in one or more other sections thereof, at least the optically transparent section(s) of the control film being connected by a first, optically clear adhesive element with a display lying therebelow and the one or more opaque sections, at least in part, being connected by a second adhesive element with a housing of the medical ventilator lying therebelow.

2. The control and display area of claim 1, wherein the display is an LED display with a pane surface.

3. The control and display area of claim 2, wherein a touch film with a transparent electrode layer is arranged between the film and the LED display unit.

4. The control and display area of claim 1, wherein the first and/or second adhesive element is an optically clear double-sided adhesive tape with a light transmission of greater than 95%.

5. The control and display area of claim 4, wherein the optically clear double-sided adhesive tape forming the first adhesive element is arranged over a whole area between display and film.

6. The control and display area of claim 4, wherein the optically clear double-sided adhesive tape comprises an acrylate adhesive.

7. The control and display area of claim 1, wherein the adhesive element forming the first adhesive element is an optically clear adhesive which has a light transmission of greater than 95% and fills a space between the film and the display.

8. The control and display area of claim 1, wherein the control film has a multi-layered design and comprises, at least in part, polyester and/or polycarbonate as well as conductor track structures for the control elements and has a thickness of at most about 3 mm.

9. The control and display area of claim 1, wherein the control film has an integral structure and thus provides a continuous surface.

10. The control and display area of claim 1, wherein the control film is adhesively bonded, over a whole area thereof, onto a surface of the medical ventilator pointing to a user.

11. The control and display area of claim 1, wherein the control film covers more than 70% of a surface of the medical ventilator pointing to a user.

12. The control and display area of claim 1, wherein the control film covers more than 80% of a surface of the medical ventilator pointing to a user.

13. The control and display area of claim 1, wherein the control film covers an entire surface of the medical ventilator pointing to a user.

14. The control and display area of claim 1, wherein an area of the control film is at least twice the size of an area of the display.

15. A method for producing a control and display area for a medical ventilator, wherein the control and display area comprises or consists of a control film with at least one control element, the control film being optically transparent in one or more sections thereof and opaque in one or more other sections thereof, and wherein the method comprises connecting at least the optically transparent section(s) of the control film by a first, optically clear adhesive element with a display lying therebelow and connecting the opaque section(s), at least in part, by a second adhesive element with a housing of the medical ventilator lying therebelow.
